(19) United States
(12) Patent Application Publication
(10) Pub. No.: US 2009/0275098 A1
(43) Pub. Date: Nov. 5, 2009

(54) SYSTEMS AND PROCESSES FOR ENHANCED YIELD FROM FERMENTATIONS THAT CONTAIN XYLOSE

(76) Inventors: Christopher Beatty, Albany, OR (US); Stephen Potochnik, Vorvallis, OR (US); Grant Pease, Corvallis, OR (US)

Correspondence Address: WHITE-WELKER & WELKER, LLC, P.O. BOX 199, CLEAR SPRING, MD, 21722-0199, US

(21) Appl. No.: 12/434,315

(22) Filed: May 1, 2009

Related U.S. Application Data
(60) Provisional application No. 61/049,765, filed on May 1, 2008.

(51) Int. Cl. C12P 7/06

(52) U.S. Cl. 435/161

(57) ABSTRACT

A system and process for the utilization of xylose during fermentation is described. The system uses a fermenter and a separate reactor to isomerize the xylose to xylulose. The separation of the two processes allows the optimization of each process since the isomerization operates ideally in a calcium free environment near pH 7.5 while the fermentation operates ideally below a pH of 6. Control of pH is assisted by the modulation of CO2 in the fermentation medium. Xylulose is fermented to ethanol by numerous standard yeasts although other products are also possible. The separate reactor may be run in a single pass, or, more preferably in a recirculating mode to allow full isomerization while the xylulose product is being consumed by the yeast. A preferred embodiment includes a Simultaneous Saccharification and Fermentation system where the liquid portion of the fermenting broth is isomerized and returned to the fermentation vessel.

### CROSS REFERENCE TO RELATED APPLICATIONS

This application claims priority from U.S. Provisional Patent Application Ser. No. 61/049,765, entitled “SYSTEMS AND PROCESSES FOR ENHANCED YIELD FROM FERMENTATIONS THAT CONTAIN XYLOSE”, filed on 1 May 2008. The benefit under 35 USC §119(e) of the United States provisional application is hereby claimed, and the aforementioned application is hereby incorporated herein by reference.

### SEQUENCE LISTING OR PROGRAM

Not Applicable

### FEDERALLY SPONSORED RESEARCH

Not Applicable

### TECHNICAL FIELD OF THE INVENTION

The present invention relates generally to xylose fermentation processes. More specifically the present invention relates to systems and processes for enhancing the yield of fermentations that contain xylose, including production of ethanol from lignocellulosic substrates.

### BACKGROUND OF THE INVENTION

Ethanol has become an increasingly important source for motor fuel and fuel additive. Biorefining processes which convert sugars and starches to ethanol via a fermentation pathway have long been used to produce ethanol for these fuels. Commonly used feedstocks for ethanol production include corn and sugarcane because they have accessible sugars and starches that are easily fermented into ethanol. More recently, interest in the production of ethanol and other chemicals from lignocellulosic materials such as wood residues, corn stover, and various straws has risen dramatically. Hydrolysis of these materials results in a mixture of glucose, xylose, and other sugars. While the glucose ferments to ethanol in the same fashion as the sugar/starch crops, the typical Saccharomyces cerevisiae yeasts do not ferment xylose to ethanol. This invention allows for the efficient utilization of xylose with selected conventional yeasts.

### SUMMARY OF THE INVENTION

The present invention is a system and process for the utilization of xylose during fermentation. The system uses a fermenter and a separate reactor to isomerize the xylose to xylulose. The separation of the two processes allows the optimization of each process since the isomerization operates ideally near pH 7.5, 60 degrees Celsius while the fermentation operates ideally at pH less than 6, 35 degrees Celsius. Control of pH is assisted by the modulation of CO2 in the fermentation medium and/or the addition of a base such as ammonium hydroxide, urea, or sodium hydroxide. Xylulose is fermented to ethanol by numerous standard yeasts although other products are also possible. The separate reactor may be run in a single pass, or, more preferably in a recirculating mode to allow full isomerization while the xylulose product is being consumed by the yeast. A preferred embodiment includes a Simultaneous Saccharification and Fermentation (SSF) system where the liquid portion of the fermenting broth is continuously re-circulated through a separate isomerization reactor and returned to the SSF vessel.

It is an objective of the present invention to teach a process for the fermentation of xylose to ethanol.

It is another objective of the present invention to teach a process for the fermentation of xylose to ethanol that provides for the efficient utilization of xylose using selected conventional yeasts.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

FIG. 1 is a schematic, cross-sectional view of fiber bundles found in lignocellulosic substrates;

FIG. 2 is a block diagram illustrating a biorefining process used to convert lignocellulosic biomass into ethanol via a fermentation pathway in accordance with an embodiment of the present disclosure using a single-stage serial isomerization;

FIG. 3 is a block diagram illustrating a biorefining process used to convert lignocellulosic biomass into ethanol via a fermentation pathway in accordance with an embodiment of the present disclosure using a recirculating liquid in conjunction with a Simultaneous Saccharification and Fermentation system;

FIG. 4 is a graph of data documenting the pH control of the fermentation liquid in conjunction with a Simultaneous Saccharification and Fermentation system;

FIG. 5 is a data graph illustrating Xylose Utilization via Isomerization and Fermentation, and the respective concentrations of Glucose, Xylose, Xylulose, and Ethanol as the process moves though the stages of Acid Hydrolysis, Isomerization, 30 Hour Fermentation, and 124 Hour Fermentation;

FIGS. 6a and 6b are schematic drawings illustrating further details of a biorefining process used to convert lignocellulosic biomass into ethanol via a fermentation pathway in accordance with an embodiment of the present disclosure using a recirculating liquid in conjunction with a Simultaneous Saccharification and Fermentation system where the fermentation of glucose and xylose occur together in a single vessel;

FIGS. 7a and 7b are schematic drawings illustrating further details of a biorefining process used to convert lignocellulosic biomass into ethanol via a fermentation pathway in accordance with an embodiment of the present disclosure using a recirculating liquid in conjunction with a Simultaneous Saccharification and Fermentation system where only the glucose fermentation occurs simultaneously with the Saccharification. The xylose fermentation occurs in a separate vessel; and

FIG. 8 is a data graph of the progress of an isomerization reaction showing the inhibitory effect of calcium and other contaminants.

### DETAILED DESCRIPTION OF THE INVENTION

In the following detailed description of the invention and exemplary embodiments of the invention, reference is made to the accompanying drawings (where like numbers represent like elements), which form a part hereof, and in which is shown by way of illustration specific exemplary embodiments in which the invention may be practiced. These embodiments are described in sufficient detail to enable those skilled in the art to practice the invention, but other embodiments may be utilized and logical, mechanical, electrical, and other changes may be made without departing from the scope of the present invention. The following detailed description is therefore, not to be taken in a limiting sense, and the scope of the present invention is defined only by the appended claims.

In the following description, numerous specific details are set forth to provide a thorough understanding of the invention. However, it is understood that the invention may be practiced without these specific details. In other instances, well-known structures and techniques known to one of ordinary skill in the art have not been shown in detail in order not to obscure the invention.

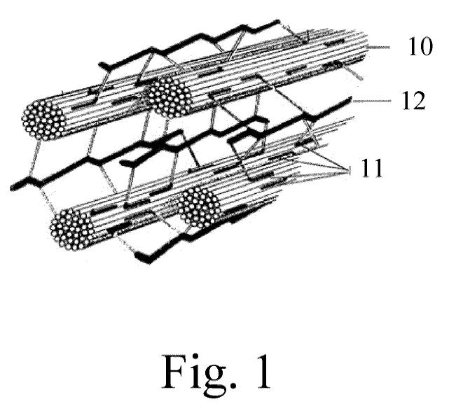

The present disclosure describes biorefining processes and systems, including processes and systems for converting lignocellulosic biomass into ethanol via a fermentation pathway. Several specific details of the disclosure are set forth in the following description and in FIGS. 1-8 to provide a thorough understanding of certain embodiments of the disclosure. One skilled in the art, however, will understand that the present disclosure may have additional embodiments, and that other embodiments of the disclosure may be practiced without several of the specific features described below.

FIG. 1 is a schematic, cross-sectional view of fiber bundles found in lignocellulosic substrates, e.g., plants. Alpha-cellulose 100 is a linear polymer of glucose that is not soluble in water. Hemicellulose 111, is a branched polymer consisting of multiple pentose (C5) and hexose (C6) sugars depending on the starting feedstock material. Xylose (not shown), a C5 sugar, is typically the most abundant sugar in hemicellulose, while arabinose, mannose, glucose, and other sugars may also be present. Lignins 112 are a cross-linking polymer that acts like a glue to hold the fiber bundles together.

Glucose, usually the most abundant C6 sugar from lignocellulosic materials, is readily fermentable by conventional yeast species such as Saccharomyces cerevisiae. Xylose, typically the most abundant C5 sugar from lignocellulosic materials, is not directly fermentable to ethanol by conventional Saccharomyces cerevisiae strains. Some wild yeast strains, such as Pichia stipitis, as well as some genetically modified yeast and bacteria strains, are able to metabolize xylose, and use of these strains in fermentation processes is an evolving aspect of biorefinement technology. Previous solutions to the utilization of xylose with these microorganisms have thus far suffered from issues with ethanol tolerance, low productivity, or metabolic imbalance.

A central concept of this invention is to convert the xylose to xylulose by an enzymatic process. These two sugars are related to each other in that xylulose is the ketose form of this C-5 sugar and xylose is the aldose form. This is the same relationship as the one between the C-6 sugars glucose (an aldose) and fructose (a ketose). The conversion between the isomers is accomplished by the same enzyme officially known as EC5.3.1.5 d-xylose ketol-isomerase or more commonly as xylose isomerase or glucose isomerase. This enzyme has been used industrially for the production of high-fructose corn syrup (HFCS) from corn (glucose) syrup since the 1970s. Unlike xylose, xylulose is converted to ethanol by several conventional yeast strains capable of high yield and ethanol tolerance including Saccharomyces cerevisiae, Saccharomyces bayanus, and Schizosaccharomyces pombe.

A challenge associated with the use of this isomerization in conjunction with fermentation is that the isomerase enzyme works optimally at a pH near 7.5 and a temperature near 60 degrees Celsius. Ethanol fermentations, particularly when performing Simultaneous Saccharification and Fermentation (SSF), as is common for lignocellulosic materials, is often carried out at a pH of about 5 and a temperature near 35 degrees Celsius. Prior art by Lastick, Smith has attempted to find “compromise” conditions that allow both reactions to occur. This has not proven to be economical because of the reduced lifetime and consumption of the isomerase. Fournier tried to overcome this issue with a bi-layer pellet that locally creates a pH environment close to the optimum for the enzyme. This has not proved economical due to the cost of the additional enzyme and does not ensure sufficient pH control to achieve adequate isomerase productivity, defined as the mass of xylulose product created divided by the mass of isomerase enzyme consumed.

A second central concept of this invention, then, is the use of a separate reactor for the isomerization process where the isomerase is immobilized and re-used. This reactor can be held at the optimum conditions for the isomerase. Two of the most important conditions are the pH (˜7.6) and a substantially calcium free environment (<5 ppm). The reactor may be a packed bed, micro channel, fiber, membrane or other style, which creates a substrate for maintaining the enzyme at optimal conditions.

Depending on the process flow and pretreatment, the isomerase reactor may be used in a serial mode with one or more stages prior to fermentation or more preferably in a recirculating mode. The isomerase reaction is a reversible equilibrium between the aldose and ketose forms of the sugar. For xylose, typical conditions result in only about 20% of the product as the more desirable xylulose. Chiang discovered that the addition of certain anions such as borate shift this equilibrium to approximately 80% xylulose. The level of borate required for high xylose conversion is compatible with fermentation. Even with this improvement, it is often desirable to recirculate the fermentation broth in order to convert the maximum amount of xylose to xylulose and eventually ethanol or other desirable products.

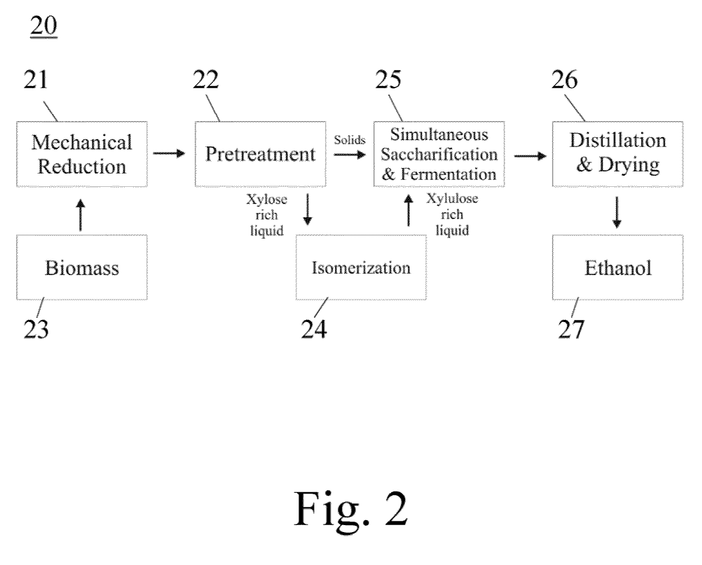

FIG. 2 is a block diagram generally illustrating a biorefining process 200 used to convert lignocellulosic biomass into ethanol via a fermentation pathway in accordance with an embodiment of the present disclosure. In a mechanical reduction process step 201, the lignocellulosic feedstock biomass 203 is brought to a facility where it is mechanically reduced by chopping, milling, grinding, cutting, etc. In a pretreatment process step 202, the biomass 203 is chemically and/or physically pre-treated to make the fiber bundles and complex polysaccharides more amenable to enzymatic hydrolysis. These secondary pretreatments can include one or more treatments with acids (e.g., sulfuric, nitric, hydrochloric), bases (e.g., NaOH, Na₂CO₃, NH₃), steam explosion, pressurized hot water, and/or solvents (e.g., acetone, ethanol), etc. to form a biomass “slurry”.

Following the pretreatment process step 202, the pre-treated biomass slurry is generally subjected to one or more enzymes (e.g., hydrolases) in a hydrolysis process step 204. The enzyme cocktail used in the hydrolysis process step breaks down the alpha- and hemicellulose polymers into fermentable sugars. Suitable enzymes can include cellulase, cellobiase, xylanase, etc. Cocktails of suitable enzymes can be purchased from Novozymes of Bagsvaerd, Denmark. In another embodiment, however, other techniques, such as acid hydrolysis, can be used to break down alpha- and hemicellulose into fermentable sugars.

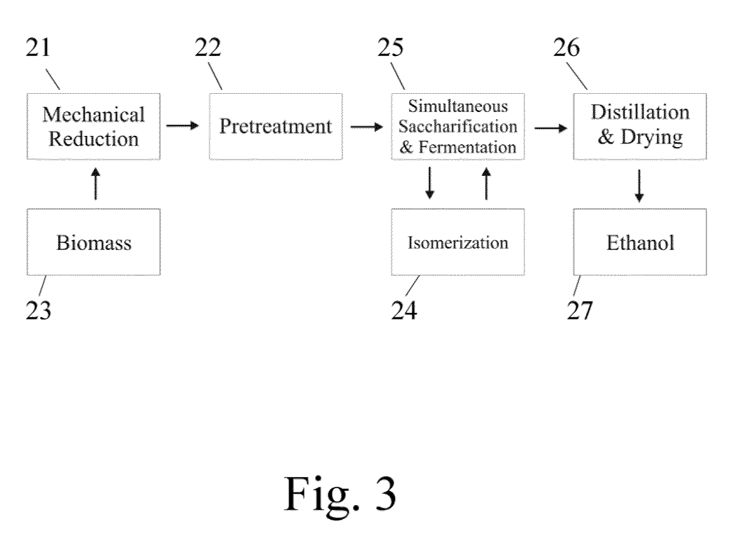

Often the enzymatic hydrolysis process step 204 is combined with a fermentation process step 205 that includes either a C6 fermentation step or both the C6 and C5 fermentation steps into a single fermentation process step 208 as shown in FIG. 3. The fermentation process produces a “beer” that is further processed in a distillation and dehydration process 206. “Beer” can be simply defined as a mix of ethanol, water, and other organics produced by the fermentation of carbohydrates. The “beer” created by the microorganisms is distilled and dehydrated 206 in much the same way as is done in the grain/corn based ethanol processes in widespread use today to produce the ethanol product 207.

A high percentage of the overall energy usage and capital cost in an ethanol plant occurs at the distillation and dehydration process. Energy usage and the associated costs are reduced with higher beer ethanol concentration. Additionally, higher beer concentration increases the throughput of ethanol for a given size of biorefinery thus lowering capital costs per unit of ethanol produced. By effectively converting xylose and glucose to ethanol, the process can increase overall ethanol yield per unit feedstock relative to the yield achieved by fermenting only the glucose.

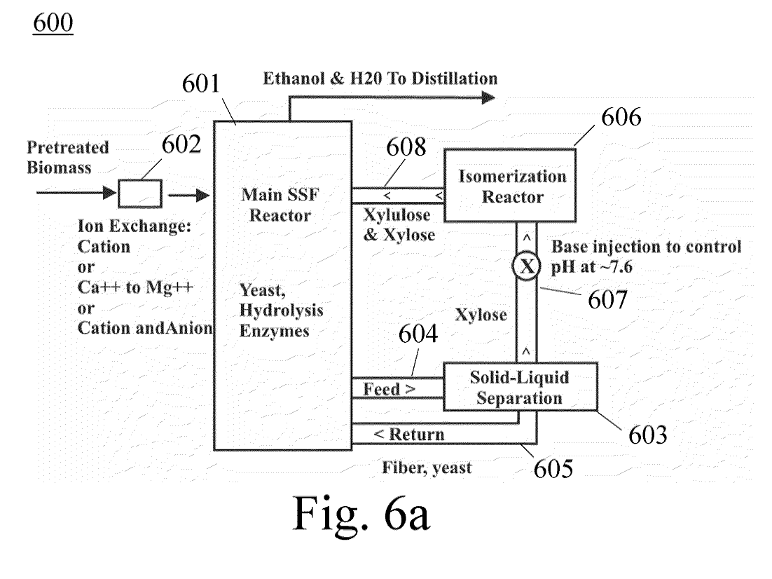

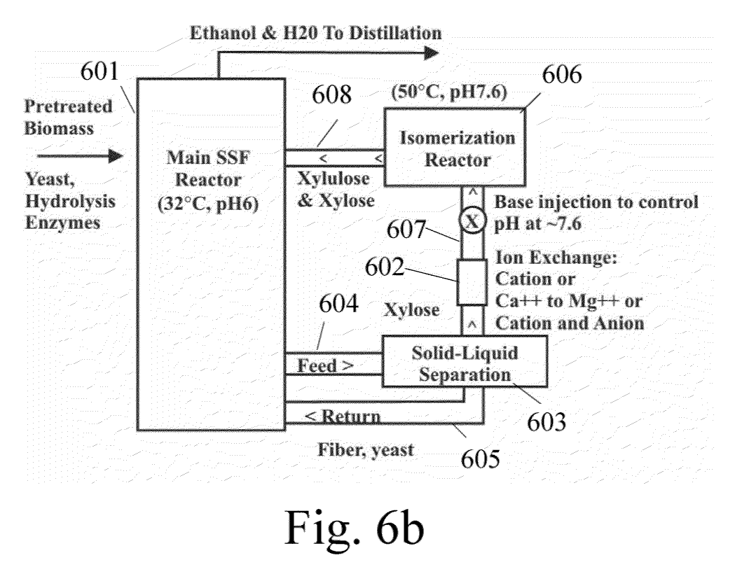

FIG. 6b is a schematic diagram illustrating additional features of the biorefining process of FIGS. 2 and 3 in accordance with an embodiment of the present disclosure. The fermenting and hydrolyzing biomass slurry is pulled from the fermentation vessel 601 and moved 604 to a solid liquid separation system 603. This may be a centrifuge, filter, or other solid liquid separation apparatus. Most of the solids and some liquid is returned 605 to the fermentation vessel 601 and the permeate or supernatant liquid is moved 607 toward the isomerization reactor 606. In order to maintain the isomerase near optimum conditions, it will often be necessary to adjust the pH and the temperature of the incoming stream of liquid 607 from the solid-liquid separation apparatus 603. Raising the pH can be accomplished partially by removing the dissolved CO2 in the liquid by “stripping” or sparging with air. This shifts the CO2/carbonate/carbonic acid equilibrium and raises the pH. Alternatively, a vacuum may be pulled on the space over the liquid to remove dissolved gases.

If further pH adjustment is required, a base such as urea, ammonia, borate, or sodium hydroxide can be added. Ammonia and urea have the added benefit of providing nutritive value for the fermentation microorganisms and avoiding the accumulation of salts in the fermentation vessel. Addition of borate anion such as sodium tetraborate will increase pH and also increase the effectiveness of the isomerase conversion by shifting the equilibrium to favor the desired product. The borate may also be added to the entire fermentation vessel instead of at this point. Shifting the temperature is optional depending on the situation since raising the temperature will increase the rate of reaction, but often lower the isomerase productivity. If heating is chosen, the temperature of the incoming liquid 607 can be altered by any heating means, although there is advantage in using a heat pump, which transfers the heat to the incoming fluid 607 and out of the fluid that has been isomerized 608 and is being cooled before being returned to the fermentation vessel 601.

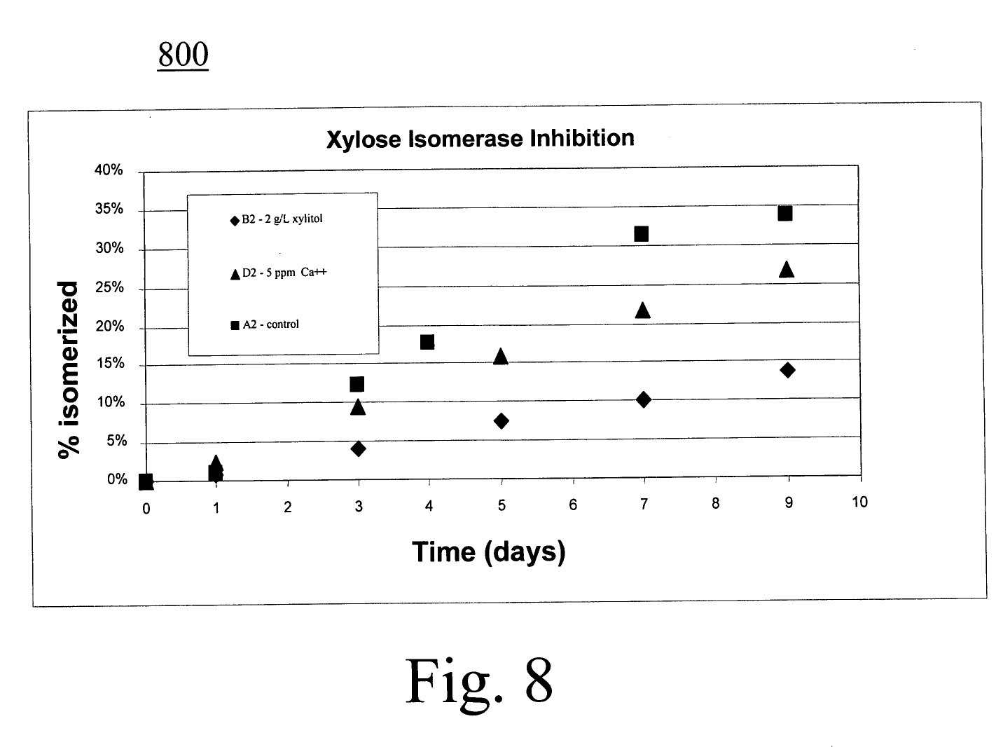

Calcium ions must be removed to avoid deactivation of the enzyme and magnesium ions, which activate the enzyme, must be added. FIG. 8 shows the inhibitory effect of calcium on the isomerization reaction. Solutions of xylose and xylose isomerase with appropriate levels of magnesium ion were intentionally “spiked” with known inhibitory compounds. The effect shown on this graph 800 is a lower rate of isomerization. The productivity of the enzyme is similarly degraded. In one embodiment shown in FIG. 6b, the calcium ions are removed 602 during the recirculation of the fermentation broth before entering the isomerization reactor 606.

U.S. Pat. No. 4,490,468 describes a xylulose fermentation system but fails to include necessary conditioning to ensure an economic lifetime of the enzyme. This may be accomplished by several means including ion exchange. In one embodiment, the ion exchange resin removes all cations including calcium. In this case, magnesium ions must be added to activate the enzyme. In another preferred embodiment, the ion exchange resin is saturated with magnesium ions before use and only the calcium ions, which bind preferentially, are removed from the fermentation broth. In yet another embodiment, the broth is fully demineralized (cation and anion removal) to maximize isomerase productivity. This may also reduce other substances such as proteins that may interfere with the activity of the isomerase. In some cases, further modifications to the liquid stream may be appropriate to condition the liquid for the isomerization reactor. Once the xylose rich liquid has been adjusted to the appropriate conditions for the isomerization reactor 606, it flows through the isomerization reactor 606 and a substantial portion of the xylose is isomerized to xylulose. Once this operation has been completed, the mixture then may be adjusted back to the conditions suitable for the fermentation. The pH is adjusted partially or completely back to the fermentation pH with the addition of acidic species. One particularly advantageous method of adding acid is to use CO2 generated by the fermentation.

By bubbling CO2 through the returning mixture, the dissolved CO2 will acidify the mixture. If this is not sufficient, other acidic species such as sulfuric acid, hydrochloric acid, phosphoric acid, or others may be used. It may not be desirable to adjust the pH completely back to the fermentation pH due to the need to offset acidification that occurs naturally during many fermentation processes. The temperature of the return stream 608 may be adjusted with cooling means such as the heat pump previously mentioned. It may be necessary or useful to overcorrect the temperature (i.e. cool below the temperature of the fermenter) due to the need to offset the heat generation that occurs in many industrial scale fermentation processes.

As mentioned before, the isomerization may be conducted serially with one or more fermentation stages or, more preferably, in a recirculating fashion that allows for a more complete isomerization of the xylose as illustrated. This occurs due to the consumption of xylulose in the fermenter such that the stream returning to the isomerization reactor is depleted in xylulose and the isomerase re-establishes the known equilibrium.

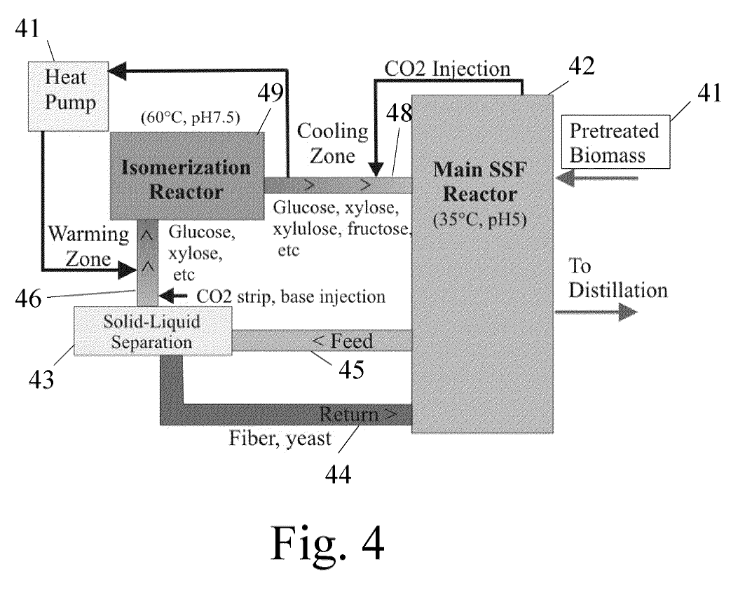

FIG. 4 is a graph 400 demonstrating the control of pH of a Simultaneous Saccharification and Fermentation broth by modulating the flow of CO2 through the material. When CO2 is purged through the vessel, the pH decreases due to CO2 forming carbonic acid in solution. When air or oxygen is purged through the vessel, the pH increases as the CO2 is brought out of solution.

The data graph 500 illustrates Xylose Utilization via Isomerization and Fermentation and the respective concentrations of Glucose, Xylose, Xylulose, Xylitol, and Ethanol as the process moves though the stages of Acid Hydrolysis, Isomerization, 30 Hour Fermentation, and 124 Hour Fermentation. The data graph 500 illustrates the change in concentration over time and at various process points as the Xylose is converted into Ethanol.

FIG. 6a is a schematic diagram showing the operations required for a successful xylose-to-ethanol system 600 that is part of a Simultaneous Saccharification and Fermentation system. In this embodiment, the fermentation of the glucose and xylose occurs in a single vessel 601. Referring specifically to FIG. 6a, the pre-treated biomass is first subjected to an ion exchanger 602 that provides for the exchange of cations or Ca++ to Mg++, or cation and anion as required. After the ion exchange is completed the biomass enters the main reactor 601 where hydrolysis and fermentation occur. During this process, a portion of the broth is sent to a solid-liquid separation area 603 via the feed tube 604. Solids are separated and sent back to the main reactor 601 via the return line 605 while liquids, such as xylose are sent to the isomerization reactor 606. During the liquid transfer, a base injection 609 to control pH at approximately 7.6 can be added to the supply line 607 before the xylose enters the isomerization reactor 606. Upon successful isomerization, xylulose and xylose are transferred back to the main reactor 601 via a return line 608.

For many feedstocks, especially agricultural residues, calcium may be an integral part of the plant tissue. In this case, the calcium removal by ion exchange or other means must be a part of the re-circulation loop shown in FIG. 6b. Otherwise, the calcium will degrade the isomerase and cause a loss of economic viability of the process. It may be possible for some feedstocks that have very low calcium in which case the calcium removal may be completed outside of the recirculation loop as shown in FIG. 6a. While this is desirable, it is only possible for very low calcium feedstocks.

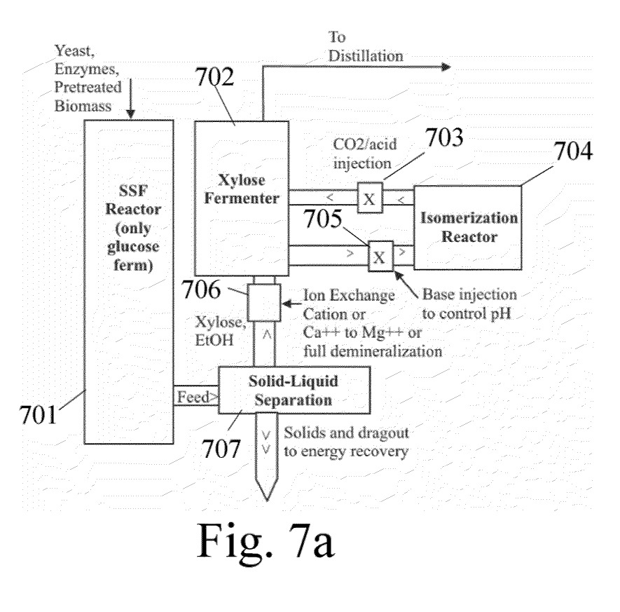

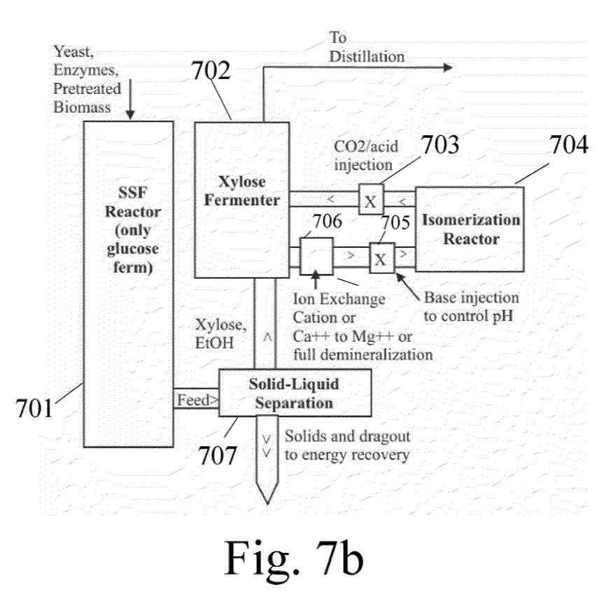

FIGS. 7a and 7b are schematic diagrams showing the operations required for a successful xylose-to-ethanol system that is part of a Simultaneous Saccharification and Fermentation system. In this embodiment, the fermentation of the glucose and xylose occurs sequentially in two separate vessels. First the yeast, enzymes, and pretreated biomass is placed in the SSF Reactor 701 for glucose fermentation only. Upon successful fermentation, the biomass is feed to the solid-liquid separation chamber 707 where solids are removed to recovery and the liquid is transferred to the xylose fermenter 702. During the transfer of the liquid from the solid-liquid separation chamber 707 to the xylose fermenter 702, the liquid is subjected to an ion exchange 706. After successful glucose fermentation the product is transferred to the isomerization reactor 704. During the transfer from the xylose fermenter 702 to the isomerization reactor 704 the mixture is subjected to a base injection 705 to control pH. After isomerization is complete the mixture is transferred back to the xylose fermenter 702. During the transfer from the isomerization reactor 704 to the xylose fermenter 702 the mixture is subject to a carbon dioxide or acid injection. This process can be repeated until xylose fermentation is complete and the mixture is moved to distillation.

The bulk of the calcium can be removed before the main process if the yeast and other parts of the system do not introduce calcium as shown in described in FIG. 7a. If this is not the case and an abundance of calcium is in the system, then the calcium removal by ion exchange or other means must be in the recirculation loop shown in FIG. 7b. In this embodiment, the ion exchange 706 occurs during xylose fermentation when the product is transferred to the isomerization reactor 704. During the transfer from the xylose fermenter 702 to the isomerization reactor 704 the mixture is subjected first to an ion exchange 706 and then a base injection to control pH 705 before entering the isomerization reactor 704.

### EXAMPLE 1

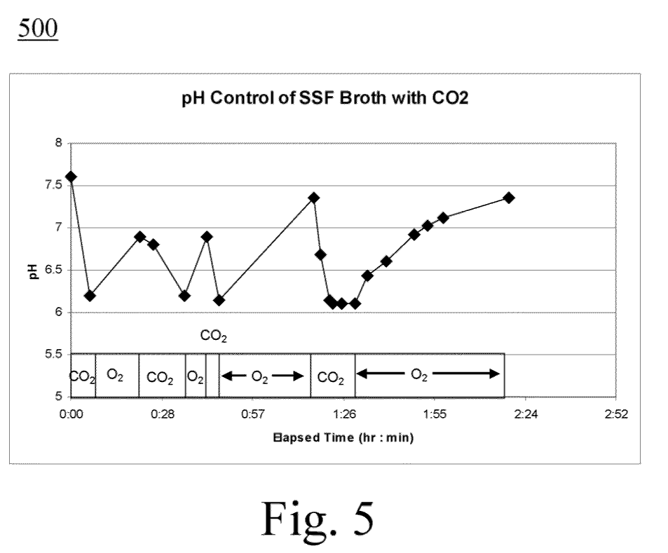

100 grams of washed ryegrass (Lolium) straw was partially hydrolyzed in 0.75 wt % H2SO4 for one hour at 121 degrees Celsius. The resulting liquor had approximately 13.7 g/l of monomeric xylose. A small amount of glucose was also present (˜1.9 g/l). The pH was adjusted to 7.6 with sodium hydroxide and 0.01M sodium tetraborate was added to shift the equilibrium toward the desired xylulose product. The liquor was isomerized in a single pass to yield about 4.2 g/l xylose and 9.5 g/l of xylulose. The pH was lowered to 5.7 and fermented at 35 C using a conventional Saccharomyces cerevisiae yeast. The resulting ethanol concentration was 4.8 g/l. Nearly all of the xylose was gone, although a small amount of xylulose remained unfermented. Approximately 35% of the estimated xylose mass in the straw was converted to ethanol mass after the fermentation. The concentrations of important chemical species are graphed in FIG. 5.

Thus, it is appreciated that the optimum dimensional relationships for the parts of the invention, to include variation in size, materials, shape, form, function, and manner of operation, assembly and use, are deemed readily apparent and obvious to one of ordinary skill in the art, and all equivalent relationships to those illustrated in the drawings and described in the above description are intended to be encompassed by the present invention.

In addition, other areas of art may benefit from this method and adjustments to the design are anticipated. Thus, the scope of the invention should be determined by the appended claims and their legal equivalents, rather than by the example given.

1. A process for the utilization of xylose during fermentation comprising:
a xylulose intermediate product;
yeast;
a fermenter;
said fermenter operating under conditions for a pH ranging from 3 to 6;
fermenting the xylulose intermediate product;
a separate isomerization reactor for isomerizing xylose to xylulose;
said isomerization occurring under conditions for a pH ranging from 7 to 8;
a solid-liquid separator for separating the solid components of the fermented xylulose intermediate product and sending the liquid components of the fermented xylulose intermediate product to an isomerization reactor; and
an ion exchanger providing removal of calcium from the fermented and isomerized product.

2. The process for the utilization of xylose during fermentation of claim 1 wherein the process is run in a single pass.

3. The process for the utilization of xylose during fermentation of claim 1 wherein
the process is run in a recirculating mode; and
wherein the fermented and isomerized product is subject to the process a plurality of times to allow maximum isomerization while the xylulose product is being consumed by the yeast.

4. The process for the utilization of xylose during fermentation of claim 1 wherein the fermentation occurs under fermentative conditions ranging from about 20-40 degrees C. in the fermenter.

5. The process for the utilization of xylose during fermentation of claim 1 wherein the isomerization occurs under conditions ranging from about 20 degree. C. to 70 degrees C. in the isomerization reactor.

6. The process for the utilization of xylose during fermentation of claim 1 wherein control of pH is assisted by a modulation of carbon dioxide in the fermentation medium between the fermenter and the isomerization reactor.

7. The process for the utilization of xylose during fermentation of claim 1 wherein calcium removal occurs by an ion exchange resin.

8. The process for the utilization of xylose during fermentation of claim 7 wherein cation removal occurs by ion exchange.

9. The process for the utilization of xylose during fermentation of claim 7 wherein Ca++ to Mg++ occurs by ion exchange.

10. The process for the utilization of xylose during fermentation of claim 7 wherein cation and anion removal occurs by ion exchange.

11. The process for the utilization of xylose during fermentation of claim 1 wherein calcium removal occurs by selective chelation of calcium ions and not magnesium ions.

12. The process for the utilization of xylose during fermentation of claim 1 wherein CO2 is bubbled through either the solids rich stream from solid-liquid separation or the xylulose rich stream from the isomerization reactor being returned to the fermenter, said carbon dioxide being dissolved into the liquid to acidify the mixture.

13. The process for the utilization of xylose during fermentation of claim 12 further requiring the addition of other acidic species such as sulfuric acid, hydrochloric acid, or phosphoric acid.

14. The process for the utilization of xylose during fermentation of claim 1 wherein the temperature of the either the solids rich stream from solid-liquid separation or the xylulose rich stream from the isomerization reactor being returned to the fermenter by the solid-liquid separator is adjusted.

15. The process for the utilization of xylose during fermentation of claim 14 wherein the temperature modulation means is a heat pump or heat exchanger.

16. The process for the utilization of xylose during fermentation of claim 15 wherein
the heat pump or heat exchanger transfers heat to the liquid components of the fermented xylulose intermediate product being sent to the isomerization reactor by the solid-liquid separator; and
transfers heat from the solid rich stream of the fermented xylulose intermediate product being returned to the fermenter by the solid-liquid separator or from the liquid stream exiting the isomerization reactor.

17. The process for the utilization of xylose during fermentation of claim 1 where the temperature of the solid components of the fermented xylulose intermediate product being returned to the fermenter by the solid-liquid separator is adjusted with cooling means below the temperature of the fermenter.

18. The process for the utilization of xylose during fermentation of claim 1 where the temperature of the xylulose rich liquid stream exiting the isomerization reactor is adjusted with cooling means below the temperature of the fermenter.

19. The process for the utilization of xylose during fermentation of claim 1 wherein the isomerization may be conducted serially with one or more fermentation stages.
